(19) United States
(12) Patent Application Publication
(10) Pub. No.: US 2023/0285082 A1
(43) Pub. Date: Sep. 14, 2023

(54) HIP ARTHROPLASTY PLANNING METHOD

(71) Applicant: Formus Labs Limited, Auckland (NZ)

(72) Inventors: George Grammatopoulos, Ottawa (CA); Ju Zhang, Auckland (NZ)

(21) Appl. No.: 18/117,109

(22) Filed: Mar. 3, 2023

Related U.S. Application Data
(63) Continuation of application No. PCT/NZ2021/050155, filed on Aug. 30, 2021.

(30) Foreign Application Priority Data
Sep. 4, 2020 (NZ) 767569

(51) Int. Cl. A61B 34/10; A61F 2/34; A61F 2/46; G16H 50/30

(52) U.S. Cl. CPC A61B 34/10; A61F 2/34; A61F 2/4607; G16H 50/30; A61F 2002/4633

(57) ABSTRACT

A method of planning a hip arthroplasty for a subject is described the method comprising: receiving at least one image of the subject, wherein the at one least image is substantially acquired from the sagittal plane when the subject is standing; calculating at least one spinopelvic metric from the at least one image; and based on the calculated spinopelvic metrics, calculating a hip arthroplasty risk characteristic of the subject. Also described are a method of determining a range of acetabular cup orientation angles for an acetabular cup implant for a subject is also described and a computer-implemented method of determining a range of orientation angles for an acetabular cup implant for a subject.

### CROSS-REFERENCE TO RELATED APPLICATION

This is a bypass continuation application of International PCT Application No. PCT/NZ2021/050155 filed on Aug. 30, 2021, which claims priority to New Zealand Patent Application No. 767569, filed on Sep. 4, 2020, which are incorporated by reference herein in their entirety.

### FIELD

This invention relates to a method of planning a hip arthroplasty. In other examples the invention relates to a method of determining a range of orientation angles for an acetabular cup.

### BACKGROUND

Hip arthroplasty is a surgical procedure in which a prosthetic is implanted in a subject to replace a hip joint. The prosthetic typically emulates the natural ball and socket hip joint by way of a femoral head that rotates within an acetabular cup.

In some cases, subjects experience pain, dislocation or other unsatisfactory functioning of the hip joint after surgery. This can lead to ongoing problems and in some case requires revision surgery.

Surgeons may endeavour to install an acetabular cup within a range of angles generally considered to provide acceptable results. However, even within this range subjects may experience problems such as pain or dislocation.

### SUMMARY

According to one example there is provided a method of planning a hip arthroplasty for a subject, comprising: receiving at least one image of the subject, wherein the at one least image is substantially acquired from the sagittal plane when the subject is standing; calculating at least one spinopelvic metric from the at least one image; and based on the calculated spinopelvic metrics, calculating a hip arthroplasty risk characteristic of the subject.

According to another example there is provided a method of determining a range of acetabular cup orientation angles for an acetabular cup implant for a subject, comprising: receiving at least one image of the subject, wherein the at one least image is substantially acquired from the sagittal plane when the subject is standing; receiving a range of anteversion angles; receiving a range of inclination angles; determining a range of acetabular cup orientation angles for an acetabular cup implant for a subject based on the calculated spinopelvic metrics, received range of anteversion angles and the received range of inclination angles.

According to another example there is provided a computer-implemented method of determining a range of orientation angles for an acetabular cup implant for a subject, comprising: receiving a range of anteversion angles; receiving a range of inclination angles; receiving a spinopelvic metric of the subject; and determining a range of acetabular cup orientation angles for an acetabular cup implant for a subject.

It is acknowledged that the terms “comprise”, “comprises” and “comprising” may, under varying jurisdictions, be attributed with either an exclusive or an inclusive meaning. For the purpose of this specification, and unless otherwise noted, these terms are intended to have an inclusive meaning—i.e., they will be taken to mean an inclusion of the listed components which the use directly references, and possibly also of other non-specified components or elements.

Reference to any document in this specification does not constitute an admission that it is prior art, validly combinable with other documents or that it forms part of the common general knowledge.

### BRIEF DESCRIPTION OF THE DRAWINGS

The accompanying drawings which are incorporated in and constitute part of the specification, illustrate embodiments of the invention and, together with the general description of the invention given above, and the detailed description of embodiments given below, serve to explain the principles of the invention.

FIG. 1 is a flow diagram of a method according to one example.

FIG. 2 is a flow diagram of a method according to another example.

FIG. 3 is a flow diagram of a method according to another example.

FIG. 4 is an image of a standing subject in a sagittal plane showing exemplary spino-pelvic metrics.

FIG. 5 is a is an image of a deep-seated subject in a sagittal plane showing exemplary spino-pelvic metrics.

FIG. 6 is an image of a subject in a coronal plane showing an installed hip prosthesis.

FIG. 7 is an image of a subject in a sagittal plane showing an installed hip prosthesis.

FIG. 8 is a graph of a relationship between acetabular cup inclination and ante-inclination for a range of anteversion values.

FIG. 9 is a graph of a relationship between acetabular cup anteversion and ante-inclination for a range of inclination values.

FIG. 10 is a graph of a relationship between combined sagittal index and dislocation frequency.

FIG. 11 is a graph of a relationship between pelvic incidence minus lumbar lordosis and dislocation frequency.

FIG. 12 is a graph of acetabular cup anteversion and inclination for subjects having various combined sagittal index values.

FIG. 13 is a nomogram for determining acetabular cup orientation angles according to one example.

FIG. 14 is a nomogram for determining acetabular cup orientation angles according to another example.

FIG. 15 is a flow chart of an exemplary method according to another example.

FIG. 16 is a flow chart of part of the method of FIG. 15 according to one example.

### DETAILED DESCRIPTION

The methods described herein make use of patient-specific information to assess risks and determine optimal acetabular cup orientations for specific patients. The inventor has found that patient-specific spino-pelvic metrics are associated with the likelihood of a prospective arthroplasty recipient suffering negative outcomes such as a dislocation. The inventor has also found that patient-specific spino-pelvic metrics can place additional constraints on suitable installation orientations for acetabular cups in addition to universal (i.e. not patient-specific) orientation constraints that surgeons may otherwise work within. The additional patient-specific constraints may indicate a smaller range of particularly suitable acetabular cup orientations for the patient in question. The size of this smaller range may be another indicator of risk.

The spinopelvic metrics may be obtainable from one or more sagittal plane images of a standing patient. The term “sagittal plane” and the discussion of an image taken of the sagittal plane are not intended to require exact compliance with the nominal sagittal plane orientation or position on a subject. Due to variations in anatomy, imprecisions in a subject's posture during imaging and other factors this is rarely achievable. Instead, this terminology refers to images taken of a patient generally from the side of their body and image planes generally aligned with a plane passing through the anterior and posterior of a subject.

By assessing the risk to patients at an individual level, patients can be categorised by their risk level. This means that higher-risk patients can be provided with more comprehensive pre-operative imaging and analysis in preparation for surgery, whereas lower-risk patients would not need such extensive pre-operative procedures. This may decrease overall costs and improve efficiency and outcomes by directing resources to the patients most in need of them.

By taking into account patient-specific constraints on acetabular cup installation orientations, installation angles can be better tailored to individual patients. This may lead to improved surgical outcomes.

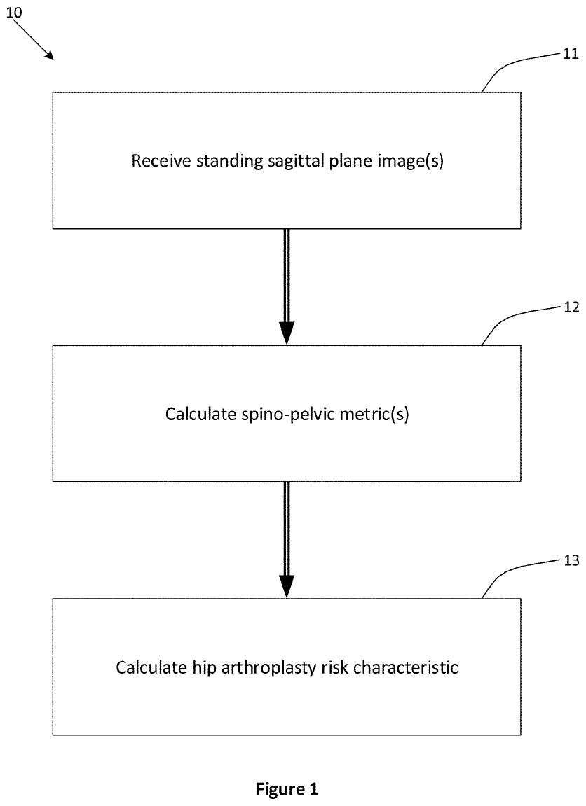

FIG. 1 illustrates an exemplary method 10 of assessing a risk associated with a prospective hip arthroplasty. The method 10 includes receiving an image of a subject, the image being taken in the sagittal plane of the subject 11. The image may be one of various suitable kinds of image. The image can include representations of the subject's skeletal structure, for example of the spine, pelvis and femur. In one example, the image is an X-ray image. Alternatively, the image may be obtained by other forms of radiography—such as gamma ray radiography—or by ultrasonic imaging or other suitable techniques capable of imaging bone. In other examples, it may be possible to use other imaging techniques that do not directly image bone if the orientation and/or position of bones can be inferred from the image.

Based on the image, one or more spino-pelvic metrics is then calculated 12. Spino-pelvic metrics relate to geometric features of the skeletal structure including the spine, pelvis and femur. Spino-pelvic metrics can be indicators of a subject's kinematics and balance. The metrics can include, for example, lumbar lordosis (LL), pelvic incidence (PI), pelvic tilt (PT), sacral slope (SS), acetabulum ante-inclination (AI) and pelvic-femoral angle (PFA) as set out in the table below.

TABLE 1

| Parameter | Measure of: |
|---|---|
| Lumbar | Degree of lumbar lordosis. The change in LL angle |
| Lordosis | reflects degree of motion; lumbar flexion (LL reduces) |
| (LL): | and extension (LL increases) |
| Pelvic | Individual pelvic morphology, which is position |
| Incidence | independent and reflects the relative anatomic position of |
| (PI): | the hip joint to the sacrum/spine. The greater the PI angle, |
| | the more anterior the hip joint is relative to the sacrum. |
| Pelvic | Sagittal position of the hip relative to the middle of the |
| Tilt (PT): | sacral endplate. The change of PT between different |
| | positions reflects the amount of pelvic tilt. |
| Sacral | The angle of the sacral endplate relative to the horizontal; |
| slope (SS): | in-part determines the positions of the lumbar spine. The |
| | change in SS with different positions is the same as the |
| | PT change. |
| Ante- | Sagittal orientation of the acetabulum or acetabular cup |
| Inclination | |
| (AI): | |
| Pelvic- | Position of the femur relative to the sacral end-plate. The |
| Femoral | smaller the angle in the standing position, the more |
| Angle | pronounced a fixed flexion contracture of the hip is. The |
| (PFA): | change in PFA between positions reflects hip flexion. |

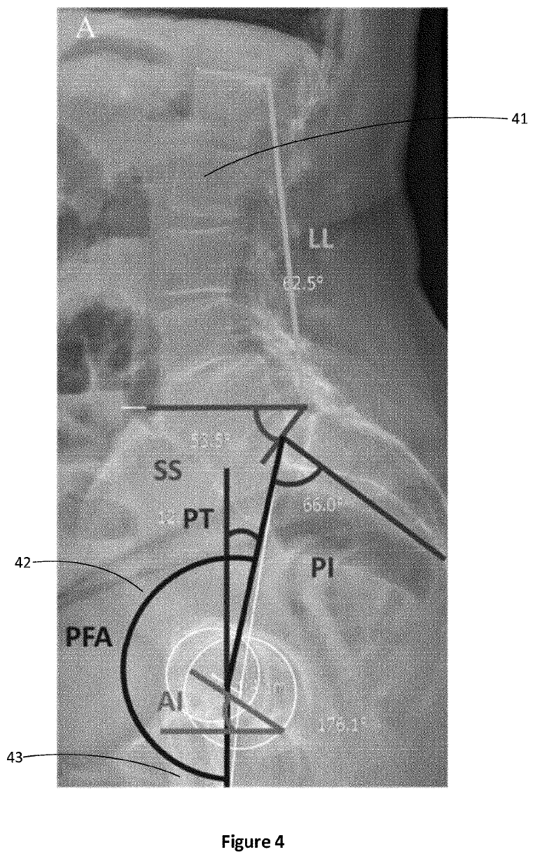

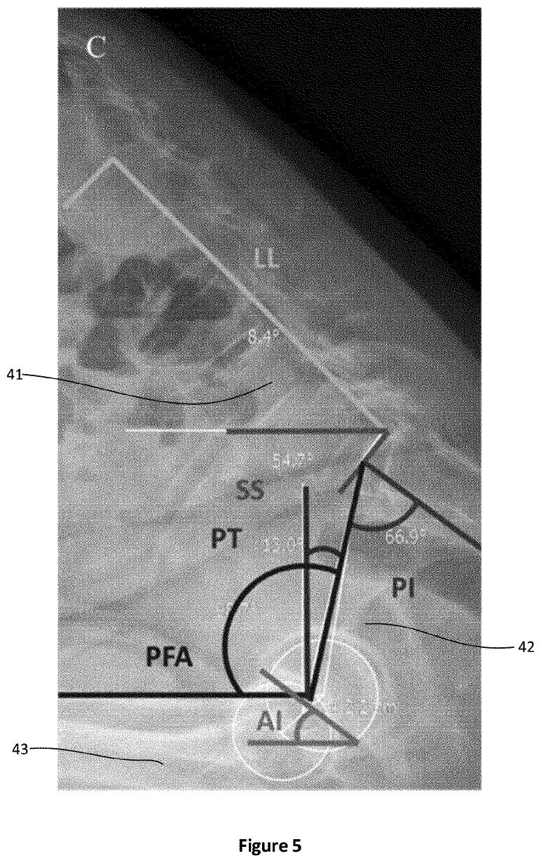

These metrics are illustrated in FIGS. 4 and 5. FIG. 4 is an X-ray image of a standing subject taken in the sagittal plane. FIG. 5 is an X-ray image of a deep-seated patient taken in the sagittal plane. The metrics are constructed based on the geometry of the spine 41, pelvis 42 and femur 43 in the respective postures.

The metrics can be measured based on anatomical landmarks. The landmarks can be identified based on user inputs. For example, a user can review the image on a computing device and place markers on the relevant landmarks. Alternatively, the landmarks may be automatically located using computer software. The computer software could include object recognition and labelling algorithms to identify landmarks and calculate the metrics. The computer software could be an artificial intelligence system. In one example, the artificial intelligence system is based on a machine learning model that has been trained on a data set related to the particular surgeon in order to learn that surgeon's preferences or techniques.

Combinations of these metrics can be used to construct other metrics for use in the risk assessment method 10. For example, sagittal balance is defined as PI−LL. As shown in FIG. 11, patients who experienced post-arthroplasty anterior or posterior dislocations tended to have higher values of PI−LL than those who did not experience dislocations. In this graph, bars 101 show frequency of dislocation (or no dislocation) as a function of PI−LL. Also, the same metric may be measured in different postures and a combination of the values of that metric in the different postures used as a metric for risk assessment. For example the difference in values of LL (ΔLL) between a standing and a deep-seated posture may be useful for determining stability or instability.

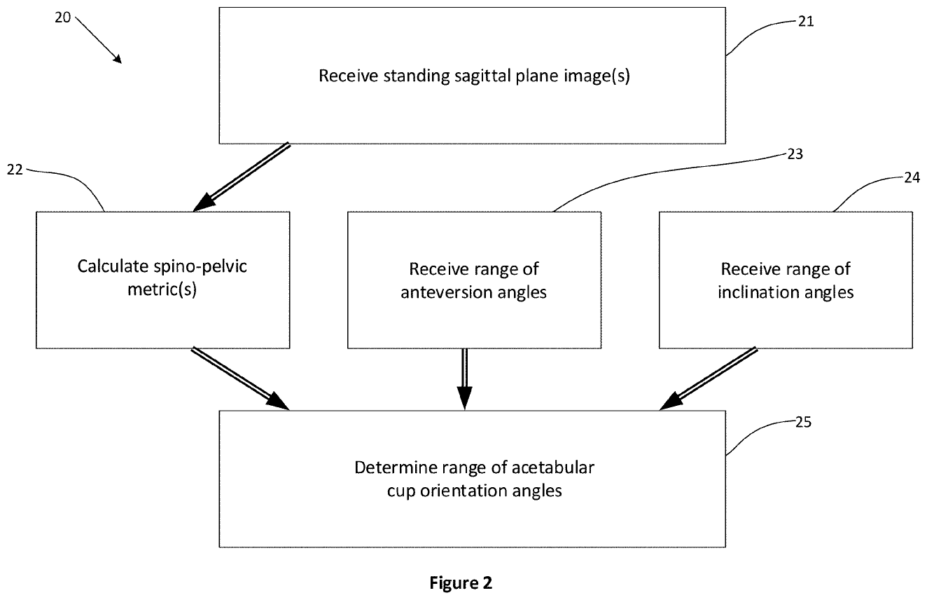

A hip arthroplasty risk characteristic of the subject is then calculated from the metrics 13. As noted above, the metrics may be used individually or in combination in determining the risk characteristic. A risk characteristic could take a range of values indicating a severity of risk or it could be a discrete risk categorisation such as “low risk” or “high risk”. The risk characteristic could be used on its own as a marker that the subject is at a certain risk of negative surgical outcomes. In one example, an instability metric is used to categorise a subject as high risk or low risk. The instability metric can be sagittal balance (PI−LL). In one example, risk categorisation is based on a subject having a value of PI−LL significantly greater than 0°, or greater than about 2°, or greater than about 10°. Subjects with PI−LL in these ranges can be categorised as being high risk. These subjects may be at an increased risk of suffering a dislocation after a hip arthroplasty. Another instability metric that could be used is ΔLL. The risk categorisation can be based on a subject having a value of ΔLL significantly lower than 40° or lower than about 29°. Subjects with ΔLL in these ranges can be categorised as high risk. Another metric that could be used is PFA. Very large or very small values of PFA could indicate risk due to the difficulty of installing an acetabular cup at a suitable orientation. For example, if post-operative combined sagittal index (CSI) is required to be within a certain range, it may be difficult to install an acetabular cup at an orientation that would result in a post-operative CSI within the required range when PFA is very large or very small. CSI is discussed in more detail with reference to FIG. 2.

Alternatively or additionally, the metric can be used in combination with other data, such as anteversion and inclination angles, to determine a risk characteristic. The metric could be used as an input to a determination of certain arthroplasty parameters, such as a range of suitable acetabular cup orientation angles. This will be described in more detail with reference to FIG. 2.

FIG. 2 lays out a method of determining a range of orientation angles for an acetabular cup implant. The method 20 could be used on its own or in combination with the method of FIG. 1.

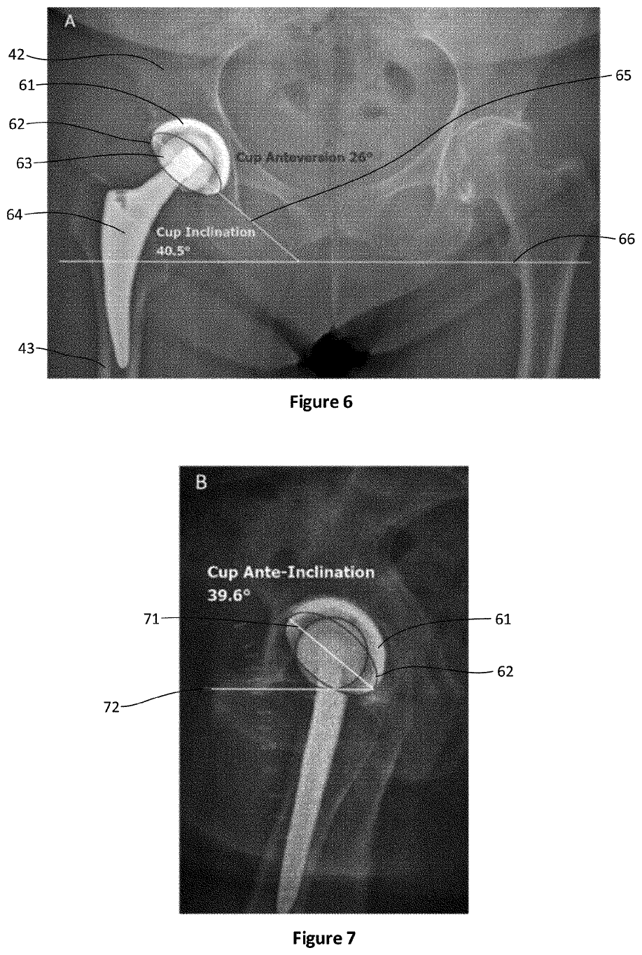

Before explaining the method of FIG. 2 in detail, relevant angles will be discussed with reference to FIGS. 6 and 7.

FIG. 6 is an X-ray image of a subject with a prosthetic hip implant 64. The image is of the coronal plane (i.e. it is taken from the front or back of the subject). The implant 64 includes a femoral head 63 and an acetabular cup 61. Together these form a ball and socket joint that emulates a natural hip. The acetabular cup 61 has a circular rim 61. The rim 62 is shown as an ellipse in the coronal plane image of FIG. 6 because it lies at an angle to the coronal plane in this example. The base of the implant 64 is installed in the subject's femur 43 and the acetabular cup 61 is installed in the subject's pelvis 42. Two widely used acetabular cup angles are shown, the radiographic inclination and radiographic anteversion. The cup inclination is the angle between the plane of the rim 62 and the subject's transverse plane (horizontal 66 in FIG. 6) when this is projected onto the coronal plane. Equivalently, it may be defined as the angle between the subject's longitudinal axis (vertical in FIG. 6) and the acetabular axis when this is projected onto the coronal plane, with the acetabular axis being the axis which passes through the centre of the acetabular cup and is perpendicular to the plane of the rim 62. The cup anteversion is the angle between the plane of the circular rim and a line orthogonal to the coronal plane. Equivalently, it may be defined as the angle between the acetabular axis and the coronal plane. This is calculated from the measured eccentricity of the image of the rim based on the knowledge that the rim is in fact circular, for example based on the relative sizes of the major and minor diameters of the elliptical image of the rim. In this example, the cup inclination is 40.5° and the cup anteversion is 26°.

FIG. 7 is an X-ray image of the subject of FIG. 6 but taken in the sagittal plane. This image shows the cup ante inclination, which is the angle between the horizontal line 72 and the straight line 71 that lies along, or is parallel to, the greatest diameter (i.e. major axis) of the image of the rim 62 in the sagittal plane. Equivalently, this may be defined as the angle between the subject's longitudinal axis (vertical in FIG. 7) and the acetabular axis when this is projected onto the sagittal plane. In this example, the ante-inclination is 39.6°.

Surgeons typically consider the coronal plane angles of anteversion and inclination when planning a hip arthroplasty. In particular, certain ranges of these angles are considered to be generally “safe” and at a low risk of negative outcomes (e.g. dislocation). However, surgeons may also exercise some judgement or personal preference for particular ranges of anteversion and inclination. However, some patients may still experience negative outcomes when acetabular cups are installed within these angle ranges due to the particular geometries of their skeletal structures. One measure of the likelihood of a patient to suffer negative outcomes is combined sagittal index (CSI). This is defined as the sum of PFA and acetabular cup ante-inclination, or CSI=PFA+AI(cup). The inventor has found that, for a given patient with a particular PFA value, some values of anteversion and inclination that might otherwise be considered “safe” in fact lead to a CSI value outside of an optimal range because anteversion and inclination are related to AI. Specifically, the inventor has determined that these are related as follows:

$${X}_{0} = \sqrt{\frac{1}{1 + {( tan β )}^{2} + \frac{1}{{( tan α )}^{2}}}}$$

$${Y}_{0} = {X}_{0} tan β$$

$${Z}_{0} = \frac{{X}_{0}}{tan α}$$

$$γ = arctan ( \frac{{Y}_{0}}{{Z}_{0}} )$$

where α is cup inclination; β is cup anteversion; and γ is cup ante-inclination.

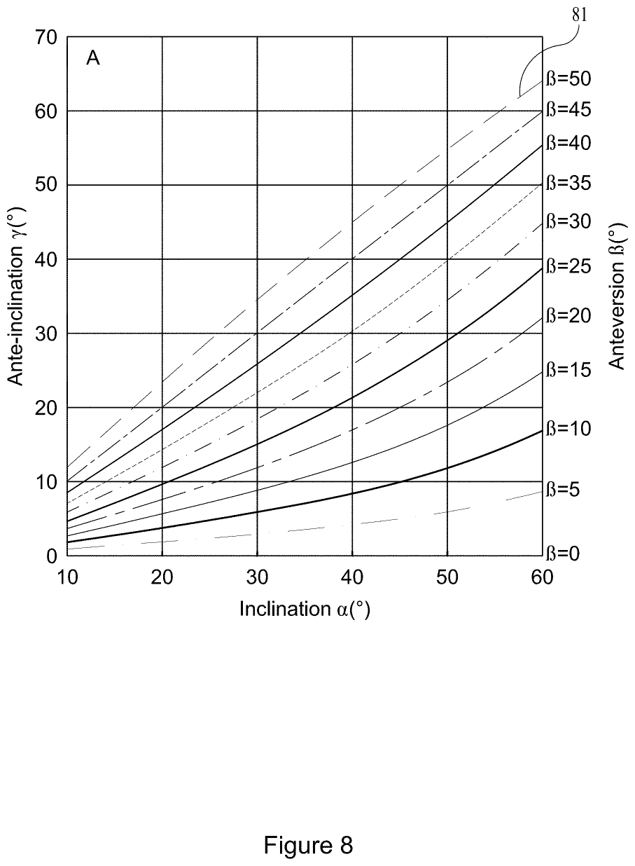

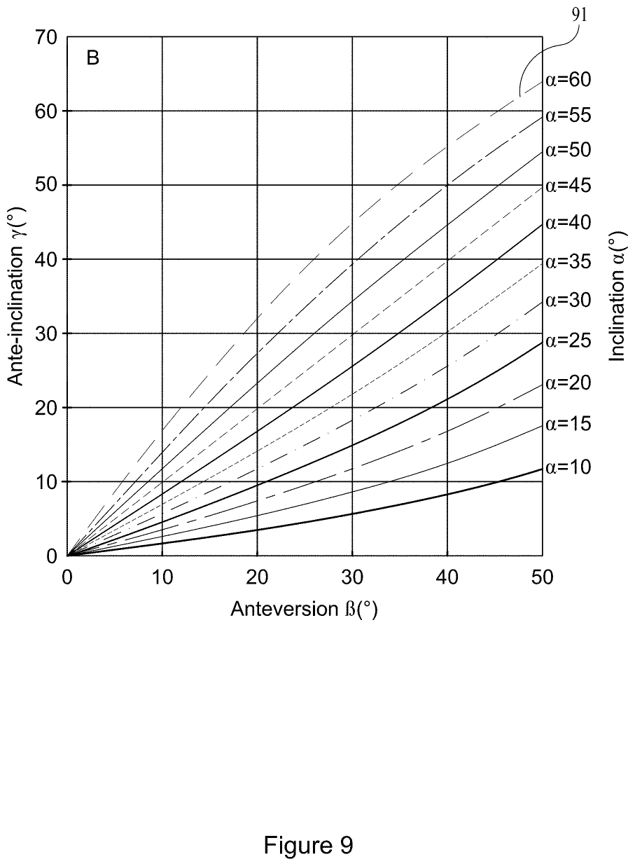

This relationship is depicted in the graphs of FIGS. 8 and 9. In FIG. 8, lines 81 represent constant-value anteversion curves. These are plotted on X and Y axes representing inclination and ante-inclination, respectively. In FIG. 9, lines 91 represent constant-value inclination curves. These are plotted on X and Y axes representing anteversion and ante-inclination, respectively.

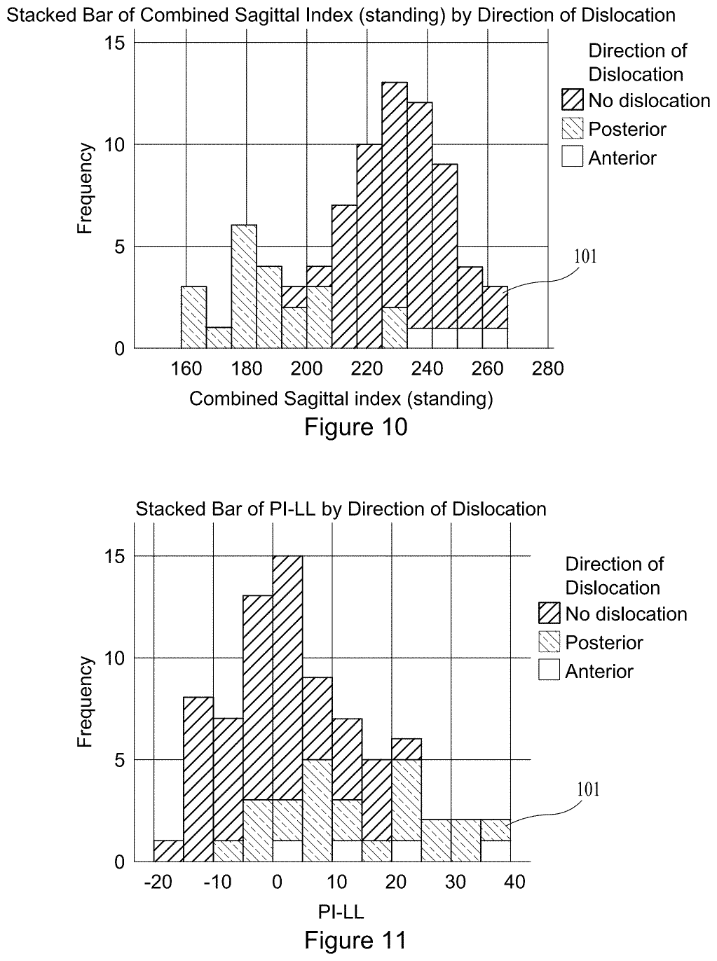

FIG. 10 shows the frequency of post-arthroplasty posterior and anterior hip dislocations as a function of standing CSI. The bars 101 represent the numbers of patients who experienced each type of dislocation (or no dislocation). As can be seen, patients who experienced anterior dislocations tended to have higher than CSI values that those who had no dislocations and patients who experienced posterior dislocations tended to have lower than CSI values than those who had no dislocations.

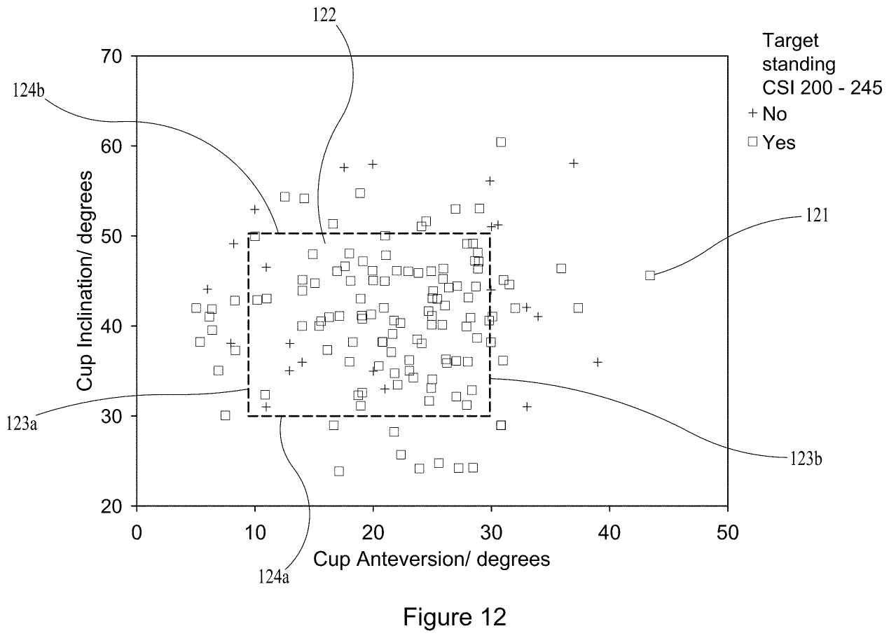

FIG. 12 is a scatter plot of cup inclination and cup anteversion of patients. The specific patient values are represented by the dots 121 and shaded according to their CSI values. Also shown is a box outlined a region 122 that corresponds to generally accepted “safe” values of inclination and anteversion angles. The region 122 is bounded by lower and upper inclination limits 124a and 124b and by lower and upper anteversion limits 123a and 123b. As will be noted, there are several patients within the region 122 that have CSI values below 200° or above 245°.

In method 20 of FIG. 2, a sagittal plane image of a standing subject is received 21. The sagittal plane image could be as described with reference to FIG. 1.

Based on the image, one or more spino-pelvic metrics are calculated 22. The metric(s) can be indicative of predicted instability of the subject post-surgery. One suitable metric for the method 20 is PFA. This may be particularly useful for placing a constraint on optimal acetabular cup installation angles.

The method 20 also includes receiving a range of anteversion angles 23. These angles may be predefined or based on the judgement or preference of a surgeon and may represent a range of anteversion angles considered to be suitable to install the acetabular cup in. In one example, the range may be centred around approximately 20°. In one example, a range of 20°±10° (i.e. 10° to 30°) is received.

The method also includes receiving a range of inclination angles 24. As with the anteversion angles, these may be predefined or based on a surgeon's judgement or preference. They represent a range of inclination angles considered to be suitable to install the acetabular cup in. In one example, the range may be centred around approximately 40°. In one example, a range of 40°±10° (i.e. 30° to 50°).

Based on the spino-pelvic metric(s), range of anteversion angles and range of inclination angles, a range of acetabular cup orientation angles is determined 25. In one example, the determined range of acetabular cup angles are angles at which the CSI of the subject would be within an acceptable CSI range if an acetabular cup were installed on that subject according to the received anteversion and inclination ranges. In one example, the acceptable CSI range is between 200°±10° and 245°±10° in a standing posture. The acceptable CSI range could be set based on empirical data, computer simulation/modelling or other research. The acceptable range could also be set based on a surgeon's skill level, preferences or judgement. The acceptable range could also be based on a level of risk tolerance or aversion. The subject's measured PFA can be used to define a range of AI(cup) values that will lead to a CSI within the acceptable range. The determined acetabular cup orientation angles can angles that are consistent with this defined range of AI(cup) values that lead to acceptable CSI values. Specifically, the AI ranges may be selected based on the equation:

CSI=PFA+ΔPFA+AI(cup)

where ΔPFA is the expected change in PFA following hip arthroplasty, such that CSI falls within the range of 200°±10°-245°±10°. ΔPFA can established clinically before surgery and typically lies within the range of 2°-10°. ΔPFA could also be given a fixed value such as 5°.

As can be seen from this relationship, the specific subject's value of PFA places a constraint on the optimal values of AI. When determining a suitable range of acetabular cup angles, the output angles can be those that meet this constraint as well as the received ranges of anteversion and inclination angles.

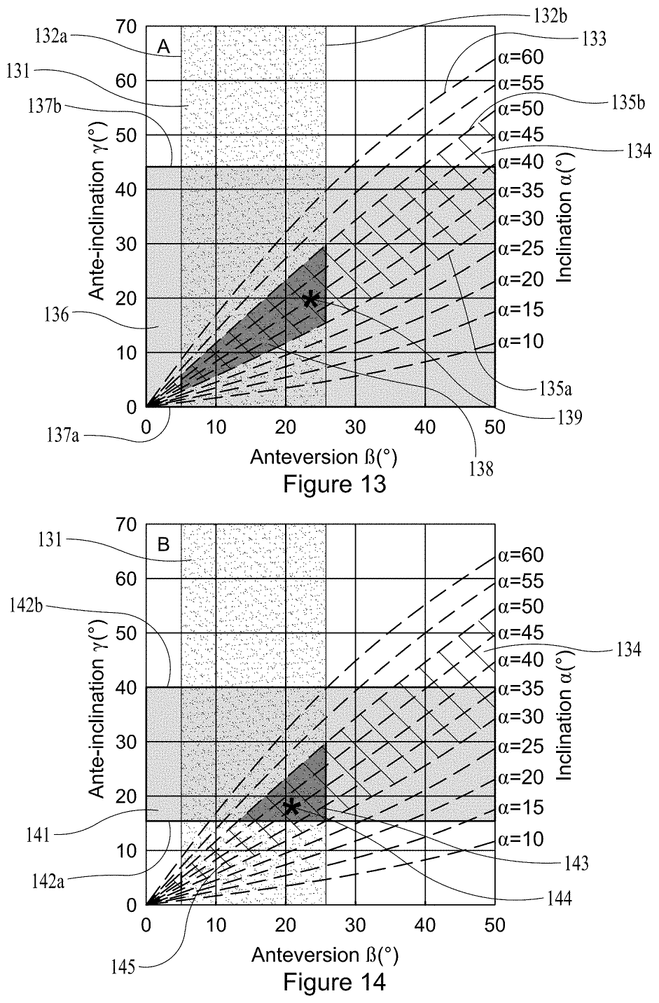

FIGS. 13 and 14 show nomograms of two different subjects A (FIG. 13) and B (FIG. 14). These nomograms depict anteversion, inclination and ante-inclination on a single plot and may be useful for determining suitable acetabular cup installation orientation angles that simultaneously satisfy anteversion, inclination and ante-inclination constraints. Lines 133 are constant-value inclination curves. Anteversion values are measured along the X axis and ante-inclination values are measured along the Y axis.

These figures show a shaded region 134 of inclination values that correspond to a received range of inclination values. This range is between a lower limit 135a and upper limit 135b. In this example, the range is between 30° and 50°. The shaded region 131 corresponds to a received range of anteversion angles. This range is between lower limit 132 and upper limit 132b. In this example, the range is between 5° and 25°. In FIGS. 13 and 14, the ranges 131 and 134 are the same for subjects A and B because these are not based on measurements of the subject.

Also shown in FIG. 13 is a shaded region 136 which corresponds to a determined range of anti-inclination (AI) values for subject A. This range is determined based on subject A's spino-pelvic metrics as detailed above. The range 136 is between lower limit 137a and upper limit 137b. In this example, the range is between 0° and approximately 44°. Region 138 of FIG. 13 corresponds to a range of acetabular cup orientation angles that satisfy all of the constraints and are suitable for acetabular cup installation. This region 138 is the region formed from the overlap of regions 134, 131 and 136 for subject A. Note that this region is subject-specific because the region 136 is based on the particular subject's spino-pelvic metric(s). Point 139 corresponds to a particular combination of cup orientation angles that is within range 138 and may be considered suitable for installation of the acetabular cup without a high likelihood of failure. In the method of FIG. 2, the determined range of acetabular values may correspond to the region 138 for subject A.

In FIG. 14, region 141 corresponds to a determined range of ante-inclination values for subject B. This region 141 is between lower limit 142a and upper limit 142b. In this example, the range is between 15° and 40° for subject B. Region 143 corresponds to a range of acetabular cup orientation angles that satisfy all of the constraints and are suitable for acetabular cup installation. This region is formed form the overlap of regions 134, 131 and 141 for subject B. Note that this region is different from, and smaller than, the region 138 in FIG. 13. Point 144 corresponds to a particular combination of cup orientation angles suitable for acetabular cup installation for subject B.

In some cases, the range of suitable angles of inclination and/or anteversion may be small based on the particular subject's spino-pelvic metrics. The size of either of these ranges may be used to determine a risk characteristic for the subject. For example, a subject for whom one or more of the ranges is small may be classified as higher risk than a subject for whom none of the ranges is small. In one example, if the determined range of either one or both of the ranges of acetabular cup inclination or anteversion is less than or equal to 10°, the subject is classified as high risk. In this example, if both ranges are greater than 10°, the subject may be classified as low risk. The size of ranges used to classify risk could be varied depending on factors such as particular surgeon's threshold for risk or taking into account other risk factors of the subject, for example. The characterisation of risk could also be proportional to the size of the ranges with multiple discrete, or a continuous scale of, risk characterisations.

Depending on the risk characterisation, the patient could be recommended for rigorous 3D planning in preparation for surgery. This may be only recommended for subjects characterised as high risk. This may involve generating 3D models of a patient's hip bones from CT or MRI scans. Alternatively, these may be reconstructed from coronal and sagittal plane X-ray data. During 3D planning, software may be used to calculate subject-specific anteversion and inclination ranges based on the patient-specific anteversion and inclination ranges calculated above; a combination of acetabular cup and stem anteversions; or a further optimised combination of anteversion and inclination angles that maximises hip range of motion. If the user customises the cup angle in the software, the software can provide warnings if the angles exceed any of the ranges above.

On the other hand, if a subject is characterised as low risk, they may be recommended for standard 2D templating.

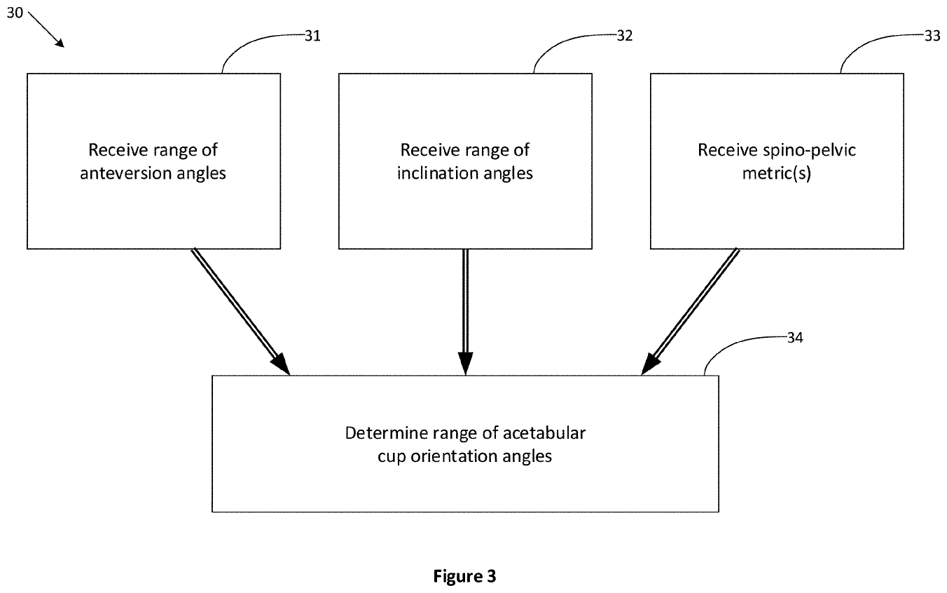

FIG. 3 shows a computer-implemented method of determining a range of acetabular cup orientation angles. In this method, a computing device receives a range of anteversion angles 31. These may be predefined or based on a surgeon's judgement of preferences as noted previously. The computing device also receives a range of inclination angles 32, which also may be predefined or based on a surgeon's judgement or preference. The computing device also receives one or more spino-pelvic metrics relating to the subject 33. Based on this information, the computing device determines a range of acetabular cup orientations 34. This can be done according to the procedures detailed above.

The computing device can be any suitable computing device having one or more interfaces for receiving and outputting information, memory and processing circuitry. In one example, the computing device is a mobile phone. The computer implement method can be performed by such a computing device operating according to a set of instructions constituting a computer programme. The computer program could be in the form of a mobile phone application.

The computer program can include instructions for performing the procedures outlined above. In particular, it can implement the methods described with reference to FIGS. 1 and 2.

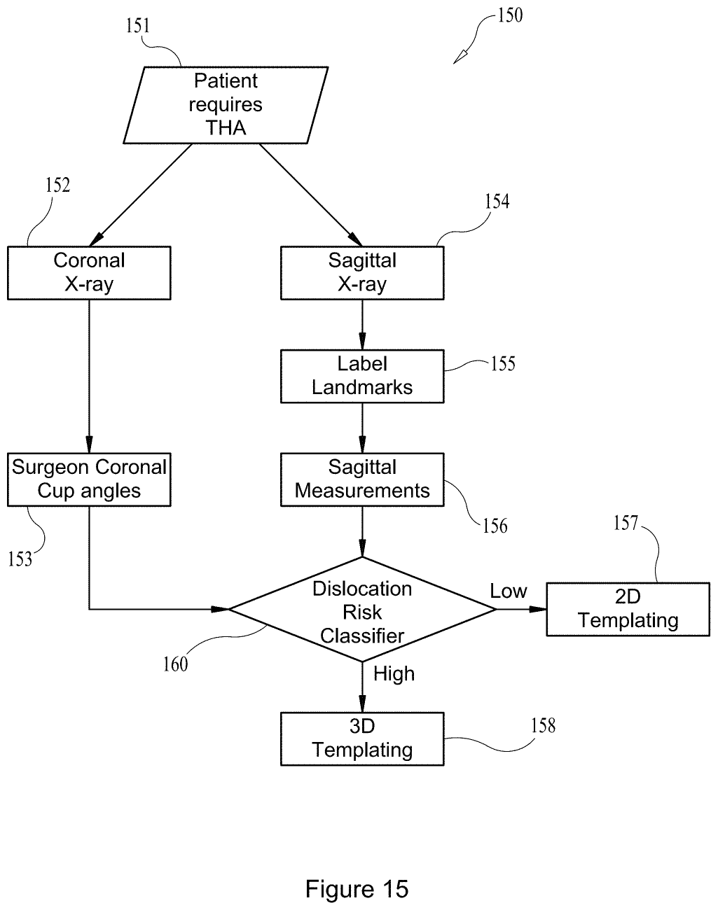

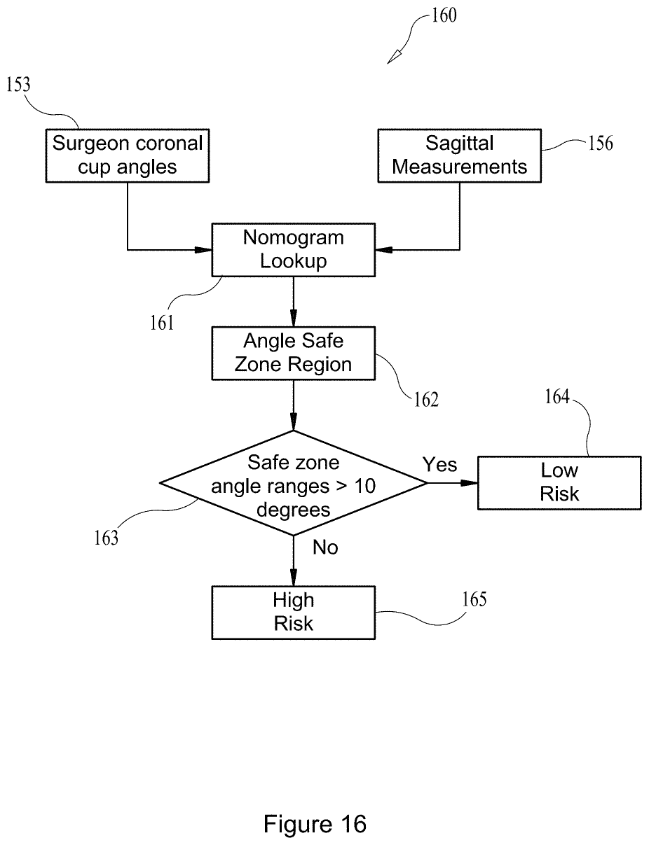

FIGS. 15 and 16 show an overview of one exemplary method 150 from arthroplasty recommendation to 2D or 3D templating. Initially, it is determined that a patient requires a total hip arthroplasty 151. A coronal x-ray is taken 152 and a surgeon determines ranges of coronal cup angles 153, which are the specified anteversion and inclination ranges detailed previously. A sagittal X-ray is also taken 154. Landmarks in the sagittal X-ray are labelled 155 and used to make measurements of sagittal spino-pelvic metrics 156 as detailed previously. The cup angles and metrics are passed to a dislocation risk classification step 160, which is shown in more detail in FIG. 16. In the subject is classified as low risk, 2D templating is performed 157. If the subject is classified as high risk, 3D templating is performed 158.

The risk classification 160 can be based on the procedures detailed previously. In particular, the surgeon's coronal cup angles 153 and the subject's sagittal measurements 156 are used are cross referenced using the nomograms 161 as detailed previously. A safe zone of acetabular cup angles is then determined 162 as the region that satisfies all of the constraints. The size of each of the ranges of determined acetabular cup angles (anteversion and inclination) are compared to a threshold value 163, in this case 10°. If both of the ranges are greater than 10°, the subject is classified as low risk 164. If one or both of the ranges is/are less than or equal to 10°, the subject is classified as high risk 165. Other threshold values may be used, for example 20°, 15° or 5°. The threshold value could be set based on empirical data, computer simulation/modelling or other research. The threshold value could also be set based on a surgeon's skill level, preferences or judgement. The threshold value could also be based on a level of risk tolerance or aversion.

While the present invention has been illustrated by the description of the embodiments thereof, and while the embodiments have been described in detail, it is not the intention of the Applicant to restrict or in any way limit the scope of the appended claims to such detail. Additional advantages and modifications will readily appear to those skilled in the art. Therefore, the invention in its broader aspects is not limited to the specific details, representative apparatus and method, and illustrative examples shown and described. Accordingly, departures may be made from such details without departure from the spirit or scope of the Applicant's general inventive concept.

What is claimed is:

1. A method of planning a hip arthroplasty for a subject, comprising:
receiving at least one image of the subject, wherein the at one least image is substantially acquired from a sagittal plane when the subject is standing;
calculating at least one spinopelvic metric from the at least one image; and
calculating a hip arthroplasty risk characteristic of the subject based on the calculated at least one spinopelvic metric.

2. The method of claim 1, further comprising:
receiving at least one image substantially acquired from the sagittal plane when the subject is sifting; and
calculating at least one spinopelvic metric from the at least one image,
wherein the hip arthroplasty risk characteristic of the subject is further based on at least the at one least image substantially acquired from the sagittal plane when the subject is standing and the at least one image substantially acquired from the sagittal plane when the subject is sifting.

3. The method of claim 1, further comprising indicating one or more proposed orientations for an acetabular cup implant based on the calculated at least one spinopelvic metric and wherein calculating a hip arthroplasty risk characteristic of the subject is further based on the one or more proposed orientations for an acetabular cup implant.

4. The method of claim 1, wherein the at least one spinopelvic metric comprises one or more of the group comprising sagittal index, sacral slope, PFA, lumbar lordosis, and overall sagittal balance.

5. The method of claim 1, further comprising calculating post-surgical standing CSI and wherein the hip arthroplasty risk characteristic of the subject is high if the calculated post-surgical standing CSI is below 200°±10° or the standing CSI is above 250°±10°.

6. The method of claim 1, further comprising calculating a range of acetabular ante-inclination angles (AI) and wherein the hip arthroplasty risk characteristic of the subject is high-risk if a calculated range of acetabular ante-inclination angles (AI) is less than 20°.

7. The method of claim 6, wherein the hip arthroplasty risk characteristic of the subject is high-risk if a calculated range of acetabular ante-inclination angles (AI) is less than 10°.

8. The method of claim 1, wherein the at least one spinopelvic metric is calculated using anatomical landmarks received from a user.

9. The method of claim 1, wherein the at least one spinopelvic metric is calculated using anatomical landmarks and wherein the method includes locating the anatomical landmarks.

10. A method of determining a range of acetabular cup orientation angles for an acetabular cup implant for a subject, comprising:
receiving at least one image of the subject, wherein the at one least image is substantially acquired from a sagittal plane when the subject is standing;
calculating at least one spinopelvic metric from the at least one image;
receiving a range of anteversion angles;
receiving a range of inclination angles; and
determining a range of acetabular cup orientation angles for an acetabular cup implant for a subject based on the calculated at least one spinopelvic metric, the received range of anteversion angles, and the received range of inclination angles.

11. The method of claim 10, further comprising:
receiving at least one image substantially acquired from the sagittal plane when the subject is sitting; and
calculating at least one spinopelvic metric from the at least one image,
wherein determining a range of acetabular cup orientation angles for an acetabular cup implant is further based on the at least one image substantially acquired from the sagittal plane when the subject is sitting.

12. The method of claim 10, wherein the at least one spinopelvic metric is calculated using anatomical landmarks received from a user.

13. The method of claim 10, wherein the spinopelvic metric comprises one or more of the group comprising sagittal index, sacral slope, PFA, lumbar lordosis, and overall sagittal balance.

14. The method of claim 10, wherein the at least one spinopelvic metric is calculated using anatomical landmarks and wherein the method includes locating the anatomical landmarks.

15. The method of claim 10, wherein the range is determined such that a calculated post-surgical standing CSI is above 200°±10° and below 250°±10°.

16. The method of claim 10, wherein the range is determined such that a calculated post-surgical standing CSI is above 200° and below 250°.

17. A computer-implemented method of determining a range of orientation angles for an acetabular cup implant for a subject, comprising:
receiving a range of anteversion angles;
receiving a range of inclination angles;
receiving at least one spinopelvic metric of the subject; and
determining a range of acetabular cup orientation angles for an acetabular cup implant for a subject based on the at least one spinopelvic metric, the received range of anteversion angles, and the received range of inclination angles.

18. The computer-implemented method of claim 17, wherein the spinopelvic metric comprises one or more of the group comprising, sagittal index, sacral slope, PFA, lumbar lordosis, and overall sagittal balance.

19. The computer-implemented method of claim 17, wherein the range is determined such that a calculated post-surgical standing CSI is above 200°±10° and below 250°±10°.

20. The computer-implemented method of claim 17, wherein the range is determined such that a calculated post-surgical standing CSI is above 200° and below 250°.
